(19) United States
(12) Patent Application Publication
(10) Pub. No.: US 2014/0072650 A1
(43) Pub. Date: Mar. 13, 2014

(54) GELATIN EXTRACT MADE FROM SKATE RAY SKIN AND ANTIHYPERTENSIVE COMPOSITION HAVING PEPTIDE ISOLATED FROM EXTRACT AS ACTIVE INGREDIENT

(71) Applicant: PUKYONG NATIONAL UNIVERSITY INDUSTRY-UNIVERSITY CO, Busan (KR)

(72) Inventors: Se-Kwon KIM, Busan (KR); Bo-Mi RYU, Busan (KR)

(73) Assignee: Pukyong National University Industry-University Cooperation Foundation, Busan (KR)

(21) Appl. No.: 13/665,690

(22) Filed: Oct. 31, 2012

(30) Foreign Application Priority Data
Sep. 12, 2012 (KR) 10-2012-0100735

(51) Int. Cl. A61K 35/60; A23L 1/29; C07K 7/00

(52) U.S. Cl. CPC A61K 35/60; C07K 7/00; A23L 1/296
USPC 424/574; 514/15.7; 435/68.1

(57) ABSTRACT

Disclosed is an antihypertensive composition containing as an active ingredient a gelatin extract made from the skins of a skate ray and a peptide isolated from the extract. The present invention is directed to a pharmaceutical composition for a hypertension prevention and therapy containing as an active ingredient a hydrolysate of a gelatin extract isolated from the skins of a skate ray, a method for preparing a hydrolysate of a gelatin extract made from the skins of a skate ray, and the use of a new peptide having an antihypertensive activity isolated and purified from the hydrolysate.

### TECHNICAL FIELD

The present invention relates to a gelatin extract made from a skate ray skin and an antihypertensive composition having a peptide purified and isolated from the extract as an active ingredient.

### BACKGROUND ART

The hypertension is a disease that 15˜20% of adults are suffering from throughout the world while causing a chronic disease such as arteriosclerosis, stroke, myocardial infarction and diabetics, which consequently results in lots of losses in terms of social and economical views. The hypertension occurs as the operation of cells serving to maintain a normal blood pressure destroys slowly and sometimes becomes a cause of circulatory system diseases.

The causes of a hypertension are not known; however there are some known factors serving to raise a blood pressure, among which a renin-angiotensin-aldosterone ring is known as adjusting a blood pressure and a body fluid in a human body. Angiotensin I which is a deactivation decapeptide is converted to angiotensin II which is octapeptie having a blood vessel contraction function as His-Leu of C-terminus detaches by means of angiotensin I converting enzyme (ACE). The thusly produced angiotensin II serves to directly contract a blood vessel and helps increase the secretion of aldosterone from a kidney, thus raising a body fluid and a blood pressure. So, it is possible to retard the rises of blood pressure by slowing the generation of angiotensin II which is a cause of the rise of a blood pressure by inhibiting the activation of ACE. The discovery of medicine serving to effectively adjust the operation of a renin-angiotensin-aldosterone ring is very important when performing a research on a hypertension therapy medicine.

As one of the representative ACE inhibitors generally used as a hypertension therapy medicine, enelapril or captopril is developed and used; however side effects such as dry cough, headache, hot flush, anorexia, taste sense disorder, rash, white blood cell reduction, etc. occur. In recent years, the development of a new ACE inhibitor which does not have side effects is urgently needed.

### DISCLOSURE OF INVENTION

Accordingly, the present invention is made to resolve the problems encountered in the conventional art. The inventors of the present invention have confirmed that a gelatin extract made from a stake skin has a good antihypertensive activation while performing a study on a new hypertension therapy medicine while providing few side effects, so the present invention is made by separating and identifying a peptide having an antihypertensive activation from the extract.

It is an object of the present invention to provide a pharmaceutical composition for a hypertension prevention and therapy which contains as an active ingredient a hydrolysate of a gelatin extract isolated from a skate ray skin.

It is another object of the present invention to provide a pharmaceutical composition for a hypertension prevention and therapy which contains as an active ingredient a peptide formed of an amino acid sequence of MVGSAPGVL (molecular weight: 829 Da) or LGPLGHQ (molecular weight: 720 Da).

It is another object of the present invention to provide a method for preparing a hydrolysate of a gelatin extract made from a skate ray skin.

It is another object of the present invention to provide a health function food for a hypertension prevention and improvement which contains as an active ingredient a hydrolysate of a gelatin extract isolated from a skate ski or a peptide formed of an amino acid sequence of MVGSAPGVL (molecular weight: 829 Da).

It is another object of the present invention to provide a hypertension therapy method using a hydrolysate of a gelatin extract and a peptide of the present invention.

To achieve the above objects, there is provided a pharmaceutical composition for a hypertension prevention and therapy, comprising a hydrolysate of a gelatin extract isolated from the skins of a skate ray, the hydrolysate being contained as an active ingredient.

According to an embodiment of the present invention, the hydrolysate is a second hydrolysate which is obtained by way of a first hydrolysis with Alcalase with respect to the gelatin extract and then by way of a hydrolysis with Protease type X.

According to an embodiment of the present invention, the gelatin extract is obtained in such a way that the skins of a skate ray are soaked in 1% of Ca(OH)₂ and is hot water extracted at pH 6.0 and a temperature of 60˜70° C. for 6˜7 hours.

According to an embodiment of the present invention, the second hydrolysate includes a peptide formed of an amino acid sequence of MVGSAPGVL (molecular weight: 829 Da) or LGPLGHQ (molecular weight: 720 Da).

According to an embodiment of the present invention, the hydrolysate is contained with a concentration of 10 ug/ml˜1500 ug/ml in the composition.

To achieve the above objects, there is provided a method of preventing and treating hypertension comprising administering to a subject in need thereof a composition comprising a hydrolysate of a gelatin extract isolated from the skins of a skate ray, the hydrolysate being contained as an active ingredient.

To achieve the above objects, there is provided a pharmaceutical composition for a hypertension prevention and therapy, comprising a peptide formed of an amino acid sequence of MVGSAPGVL (molecular weight: 829 Da) or LGPLGHQ (molecular weight: 720 Da), the peptide being contained as an active ingredient.

According to an embodiment of the present invention, the peptide is isolated from a hydrolysate of a gelatin extract isolated from the skins of a skate ray.

To achieve the above objects, there is provided a method for preparing a hydrolysate of a gelatin extract made from the skins of a skate ray having an antihypertensive activity, comprising a step for obtaining a gelatin extract from the skin of a skate ray; a step for performing a first hydrolysis by adding an Alcalase enzyme to the gelatin extract; and a step for performing a second hydrolysis by adding a Protease type X enzyme to the first hydrolysate.

According to an embodiment of the present invention, the gelatin extract is obtained in such a way that the skins of a skate ray are soaked in 1% of Ca(OH)₂ and is hot water extracted at pH 6.0 and a temperature of 60˜70° C. for 6˜7 hours.

According to an embodiment of the present invention, the second hydrolysis is performed in such a way that Protease type X vs. substrate (gelatin extract) is mixed at a ratio of 1:100 (w:w) and is reacted at pH 7.0 at a temperature of 35˜40° C. for 3 to 6 hours.

According to an embodiment of the present invention, the first hydrolysis is performed in such a way that Alcalase vs. substrate (gelatin extract) is mixed at a ratio of 1:100 (w:w) and is reacted at pH 7.0 at a temperature of 45˜55° C. for 1 to 2 hours.

According to an embodiment of the present invention, the second hydrolysate obtained by way of the second hydrolysis contains peptide the molecular weight is less than 1 kDa.

According to an embodiment of the present invention, the peptide has an amino acid sequence of MVGSAPGVL (molecular weight: 829 Da) or LGPLGHQ (molecular weight: 720 Da).

To achieve the above objects, there is provided a health function food for a hypertension prevention and improvement, comprising a hydrolysate of a gelatin extract made from the skins of a skate ray; and a peptide formed of an amino acid sequence of MVGSAPGVL (molecular weight: 829 Da) or LGPLGHQ.

According to an embodiment of the present invention, the hydrolysate is contained with a concentration of 100 ug/ml˜1500 ug/ml.

To achieve the above objects, there is provided a method for a hypertension therapy, comprising a step for medicating a hydrolysate of a gelatin extract made from the skins of a skate ray or a peptide formed of an amino acid sequence of MVGSAPGVL (molecular weight: 829 Da) or LGPLGHQ to an object except for a human being.

To achieve the above objects, there is provided a method of preventing and treating hypertension comprising administering to a subject in need thereof a peptide formed of an amino acid sequence of MVGSAPGVL (molecular weight: 829 Da) or LGPLGHQ (molecular weight: 720 Da), the peptide being contained as an active ingredient.

### Advantageous Effects

The present invention features in an antihypertensive composition which contains as an active ingredient a gelatin extract made from a stake skin and a peptide isolated from the extract. The hydrolysate of a gelatin extract isolated from a stake skin and a new peptide isolated and purified from the hydrolysate are good at an activation function of inhibiting ACE, so the hydrolysate and the new peptide can be used as the materials of a medicine for the sake of a hypertension therapy and a health functional food.

### BRIEF DESCRIPTION OF DRAWINGS

FIG. 1 is a graph which shows a result of an analysis obtained by MALDI-TOF analyzing the distributions of the molecular weights of the protein contained in a skate ray skin gelatin extract according to an embodiment of the present invention, a first hydrolysate of the extract and a second hydrolysate of the extract.

FIG. 2 is a graph of a result after an analysis on a process concentration-based ACE inhibition is performed with respect to a second hydrolysate of a stake skin gelatin extract.

FIG. 3 is a view showing a result of a measurement on a time-based heart beat and a SBP (Systolic Blood Pressure) after a hypertension rat model is fed with a second hydrolysate of a skate ray skin gelatin extract by the concentrations, and the blank represents a group that a hydrolysate of the present invention is not applied to, and P.Control represents a positive control group that a captopril which is an antihypertensive medicine is fed to.

FIG. 4 is a view showing a result of a measurement on an average blood pressure and a diastolic phase blood pressure with respect to the same group as the group of FIG. 3.

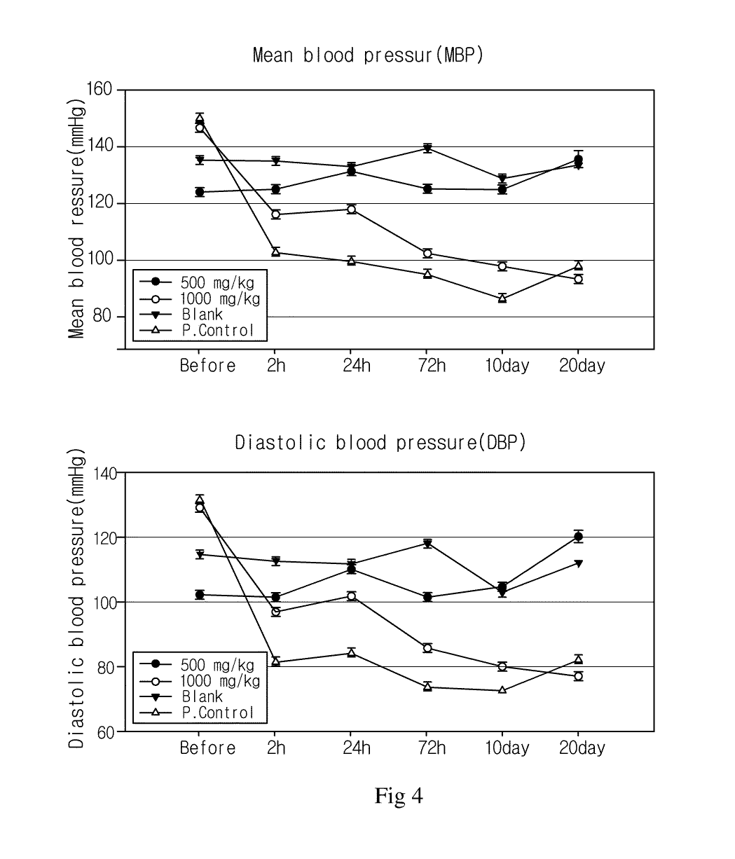

FIG. 5 is a view showing a result of the measurement on a right ventricle after a hypertension rat is fed with a second hydrolysate of a skate ray skin gelatin extract by the amount of 500 mg/kg.

FIG. 6 is a view showing a result of the measurement on an ACE inhibition with respect to a peptide isolated and purified from a second hydrolysate of a skate ray skin gelatin extract prepared according to an embodiment of the present invention.

### MODES FOR CARRYING OUT THE INVENTION

The present invention is directed to providing a pharmaceutical composition for a hypertension prevention and therapy which contains as an active ingredient a hydrolysate of a gelatin extract isolated from a skate ray skin.

Meanwhile, a skate ray is a cartilagenous fish belonging to a ray and lives in an adjacent sea of Huksan-don, Korea and a mid-south sea area of Japan. The skate ray contains lots of taurine which helps a cell membrane stabilization operation, a cholesterol adjusting operation and a function needed for the sake of physical growth and development and fatty acids such as such as linoleic acid, linolenic acid and arachidonic acid which serve to lower serum cholesterol and to enhance cognitive function. In addition, it contains EA and DHA which help enhance brain developments and eyesight enhancement functions. The bones of the skate ray contain lots of condrotin which is mucopolysaccharide protein, so they are used for the purpose of health and energetic foods while providing an antibacterial function. So, the skate ray is widely used as various food materials, antibacterial materials, etc.

However the byproducts such as the skins of the skate ray are wasted even though they contain lots of useful components.

The inventors of the present invention have obtained hydrolysate with lots of antihypertensive effects from the skins of the skate ray which used to be wasted in the past and have separated and identified from the hydrolysate two peptides which have good antihypertensive activations. It has been verified that the hydrolysate and the peptides have activations of effectively inhibiting angiotensin converting enzyme (ACE).

The ACE exists mainly in the closed capillary blood vessels and causes the contraction of the blood vessels in such a way to degrade two terminal amino acids of angiotensin I of the inactive state in the blood, thus converting to angiotensin II which raises the blood pressure by accelerating the secretion of aldosterone and increasing the body fluid, so the blood pressure rises.

In case of the hypertension therapy medicine which is currently used, the substance having an activation helping inhibit the activation of angiotensin converting enzyme has a mechanism which is directed to preventing and curing hypertension by way of the blood vessel expansion effects.

The inventors of the present invention have obtained a gelatin extract from the skins of a skate ray according to an embodiment of the present invention and have obtained a hydrolysate by processing the extract by way of an enzyme hydrolysis. As a result of the measurement on the ACE inhibition activity of the obtained hydrolysate, it has been proved that the present invention has the activity similar with the captopril which is generally used for the purpose of a hypertension therapy medicine.

Therefore, the present invention is directed to providing a pharmaceutical composition for a hypertension prevention and therapy which contains an active ingredient a hydrolysate of a gelatin extract isolated from the skins of a skate ray.

The present invention is directed to a method for preparing a hydrolysate of a gelatin extract made from the skins of a skate ray having an antihypertensive activation, and preferably the method comprises a step for obtaining a gelatin extract from the skins of a skate ray, a step for performing a first hydrolysis by adding alcalase enzyme to the gelatin extract, and a step for performing a second hydrolysis by adding protease type X enzyme to the first hydrolysate.

The method for preparing a hydrolysate of a gelatin extract made from the skins of a skate ray according to the present invention will be described below.

After a washing process is performed, the skins of the skate ray are cut at regular intervals, and the cut skate ray skins are precipitated in 1% of Ca(OH)₂ and is processed by way of a hot water extraction at pH 6.0 and 60˜70° C. for 6˜7 hours.

At this time, the skate ray skins are precipitated in 1% of Ca(OH)₂ for 2˜4 days as a process for eliminating impurities such as proteins except for subcutaneous fats and collagen. Afterward, the hot water extraction is performed in the above mentioned manner, thus obtaining a gelatin extract of the skins of a skate ray. When performing the hot water extraction out of the ranges of pH, temperature and extraction time, the active ingredients residing in the skins of a skate ray might lose activity, and the natures of it might change. For these reasons, it is preferred to perform the hot water extraction under the above mentioned conditions.

In the present invention, the term “gelatin extract” represents an extract obtained by way of the hot water extraction, and the gelatin represents a kind of protein which can be obtained while the collagen, which is a natural protein in fishes or animals, is being hot water extracted.

After the gelatin extracts are obtained from the skins of the skate ray, alcalase enzyme is added to the gelatin extract, and the step for the first hydrolysis is performed.

Here, the first hydrolysis is preferably performed in such a way that alcalase vs. substrate (gelatin extract) are mixed at a ratio of 1:100 (w:w) and are reacted at pH 7.0 and a temperature range of 45˜55° C. for 1˜2 hours. When the reaction is performed out of the above range, the optimum hydrolysis effects of enzyme cannot be obtained, and the hydrolysate obtained by way of the hydrolysis reaction does not have the targeted effects (namely, antihypertensive effects). It is therefore preferred that the first analysis is performed under the above mentioned conditions.

When the first hydrolysis is finished, the second hydrolysis is performed. The second hydrolysis is performed in such a way that the enzyme (protease type X) vs. substrate (gelatin extract) is added at a ratio of 1:100 (w:w) and they are reacted at pH 7.0 at a temperature range of 35˜40° C. for 3˜6 hours, after which the second hydrolysate can be obtained.

In the present invention, the gelatin extract obtained from the skins of a skate ray by way of the hot water extraction is processed by way of the two-step analysis. The sequential twice analysis reactions are performed to make sure that the molecular weights of the useful components contained in the skins of a skate ray are converted to low molecular weights, by means of which the useful components can be efficiently absorbed into the human body and can be easily applied to various medical medicines and foods.

In the embodiments of the present invention, the molecular weights of the components (proteins) contained in the extracts were measured with respect to the extracts processed by way of the first analysis and the extract processed by way of the second analysis. As a result of the measurements, the extracts that the analysis was not performed on had lots of high molecular weights of above 10 kDa, and the extracts processed by way of the first analysis had been proved that they had lower molecular weights as compared to the extracts not processed by way of the analysis, and lots of high molecular weights were present as compared to the extracts processed by way of the second hydrolysis. Meanwhile, it was verified that lots of molecules having lower molecular weights of below 1 kDa were present in the second hydrolysate which was processed by way of the twice hydrolysis. Consequently, it has been confirmed that in order to obtain the gelatin extracts of the skins of a skate ray containing the low molecular weights of below 1 kDa, the extracts should be processed by way of the twice analysis processes in the same manner as the above.

In addition, the inventors of the present invention have isolated and purified the active components having the antihypertensive activities with respect to the second hydrolysate which is obtained by performing a hydrolysis with respect to the gelatin extracts made from the skins of a skate ray. As a result, two peptides of MVGSAPGVL (molecular weight: 829 Da) and LGPLGHQ (molecular weight: 720 Da) are isolated and purified. In addition, as a result of the ACE inhibitions performed so as to measure the antihypertensive activities with respect to the two peptides, it has been verified that the two peptides have good ACE inhibition activities.

So, the present invention is directed to providing a pharmaceutical composition for a hypertension prevention and therapy which contains as an active ingredient the peptides formed in an amino acid sequence of MVGSAPGVL (molecular weight: 829 Da) and LGPLGHQ (molecular weight: 720 Da).

In the present invention, the peptide having an antihypertensive activity can be a peptide consisting of an amino acid sequence of MVGSAPGVL and LGPLGHQ or an acid addition salt of it. The peptide might be present in an isolated type or in a substantially pure state. Preferably, the peptide of the present invention can be a hydrolysate of a gelatin extract isolated from the skins of a skate ray, more preferably, it can be isolated and purified from the second hydrolysate.

Here, the acid addition salt might be a pharmaceutically allowable acid addition salt, and the pharmaceutically allowable acid addition salt represents salt which helps maintain the biological activity of the peptide and minimize the side effects such as unexpected toxic effects.

The acid addition salt might be a pharmaceutically allowable inorganic acid addition salt or a pharmaceutically allowable organic acid addition salt. For example, it might contain hydrochloride, brominated hydrogen acid salt, sulfate, nitrate, acetate, benzoate, maleate, fumaric acid salt, succinate, tartrate, Citrate, oxalate, methansulfonic acid salt, toluenesulfonate, aspartate or glutamate.

In addition, the pharmaceutical composition according to the present invention might contain at least one kind among the pharmaceutical diluents selected from the group consisting of salt water, buffer salt, dextrose, water, glycerol and ethanol, and it is not limited thereto.

The above mentioned composition might be adapted in different ways depending on the medication purposes and disease. The amount of the actually medicated activity components is determined in consideration of various related elements, in other words, the disease to be cured, the severities of patients, joint medications with other medicines (for example, chemotaxis medicines), ages, sexes and weights of patients, foods, medication time, medication paths and medication ratio of compositions. The above mentioned compositions can be adjusted depending on the types and severities of diseases. It can be medicated once or twice per day, but it is not limited thereto.

The composition of the present invention can be medicated via oral or parenteral paths. The parenteral medication represents, except for the oral path, a medication by way of rectum, vein, peritoneum and muscle, artery, sclerite, nasal, suction, eyes and subcutaneous medication.

The composition can be formulated in the types of an oral medication, an injection type solution or a topical formulation.

The formulation is preferably made to be in consistent with an oral or injection medication (true solution), suspension or emulsion, and most preferably, the formulation is made in an oral medication type such as tablets, capsules, smooth capsules, aqueous medicine, pills and granules.

In the middle of the formulation, the hydrolysate of the gelatin extract isolated from the skins of a skate ray of the present invention or the peptides isolated and purified from the hydrolysate can be filled in the smooth capsules without excipient being used or might be mixed with carriers or might be formulated in a proper form after dilution. As an example of a proper carrier, there are starch, water, salt water, ringer's solution, dextrose, etc.

In addition, the composition according to the present invention can be applied as a pharmaceutical composition or a food composition so as to prevent, cue or improve hypertension.

The pharmaceutical composition can be directly applied to an animal including a human being. The animal is generally fed with organic substances as nutrients in a biota corresponding to plants and has digestion and excretion and respiratory organs. It is preferably a vertebrate and is most preferably a mammal. The mammal might be a human being, a pig, a cow or a goat, and it is preferably a human being.

The pharmaceutical composition comprises a hydrolysate of a gelatin extract isolated from the skins of a skate ray or contains as an active ingredient the peptides isolated and purified from the hydrolysate. In addition, a carrier, an excipient, a diluent or a sub-component might be further included, which are pharmaceutically or nutritionally allowable depending on the formulation, use method and use purpose.

In addition to the active ingredients, the pharmaceutical composition might further comprise a nutritional supplement, vitamin, electrolyte, flavoring agent, coloring agent, filler, pectic acid and the salt of it, alginic acid and the salt of it, organic acid, protective collide thickener, pH adjuster, stabilizer, antiseptic, glycerin, alcohol and carbonization agent used for carbonated beverage. In addition, the carrier, excipient or diluent can be selected from the group consisting of lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propylhydroxybenzoate, talc, magnesium stearate and mineral oil, dextrin, calcium carbonate, propyleneglycol, liquid paraffin and saline solution. It is not limited thereto, and an ordinary carrier, excipient or diluent can be used as well.

The pharmaceutical composition might contain a hydrolysate of the present invention at a concentration of 10 ug/ml˜1500 ug/ml with respect to the total weight of the composition, and the peptide might be 0.001 weight % to 99.9 weight %, preferably, 0.1 weight % to 99 weight %, more preferably 1 weight % to 50 weight %.

In case of actual medications, the pharmaceutical composition further contains an ordinary filler, thickener, coupler, disintegration agent, surfactant, anti-cohesive agent, lubricant, moisturizer, perfumes, emulsifier or antiseptic and can be used orally or parenterally.

In more details, the solid formulation for the purpose of an oral medication comprises tablet, pill, powder, granule or capsule, and the above mentioned solid formulation can be mixed with at least one excipient, for example, starch, calcium carbonate, sucrose or lactose, gelatin, etc. In addition, in addition to a simple excipient, the lubricant such as magnesium stearate, talc, etc can be used. As the liquid formulation for the purpose of an oral medication, there are suspension, liquid medicine, emulsion, syrup, etc. In addition to a simple diluent such as water, liquid paraffin, etc., various excipient such as moisturizer, flavoring agent, perfume, preserver, etc. can be included.

In addition, the formulation of the pharmaceutical composition might change depending on the use method, and it can be formulated in a known method to make sure that a quick, continued or delayed discharge of the active component can be obtained after it is medicated to mammal. The formulation is plaster, granule, lotion, liniments, limonades, powder, syrup, eye ointment, liquid, aerosol, extracts, elixir, ointment, flow type extract, emulsion, suspension, decoctions, infusions, eye bath lotion, tablet, suppository, injection, spirits, capsule, cream, pills, and smooth or solid gelatin capsule.

In addition, the pharmaceutical composition of the present invention can be formulated with the aid of the known part and the method disclosed in the Remingtom reference.

The dose of the pharmaceutical composition of the prevention might be properly determined by an ordinary person skilled in the art in consideration of the medication method, the age, sex and weigh of the patient and the severity of the disease. For example, the pharmaceutical composition of the present invention can be dosed 0.000001 mg/kg/day to 1000 mg/kg/day with respect to the hydrolysate or the peptides. The medication might be once per day or more than once per day. The above mentioned does is not limited thereto.

Since the hydrolysate of the gelatin extract isolated from the skins of a skate ray which is an active ingredient for a hypertension therapy agent and the peptides isolated from the hydrolysate according to the present invention are made from the natural materials, they are stable in the human body.

The composition according to the present invention can be used as a food composition for the sake of prevention and therapy hypertension. As an example of the food composition of the present invention, it might be foods, food additives, beverage or beverage additives. In addition to them, a proper carrier, excipient and diluent might be further added.

The term “food” in the present invention represents a natural or processed food which contains at least one or more nutrients, preferably, represents an eatable thing somehow processed, and as an ordinary meaning, it represents foods, food additives, health functional foods and beverage, and preferably represents gum or candy.

The foods adapted to the present invention, for example, are various foods, beverage, gum, candy, tea, vitamin mixtures, functional foods, etc. The foods of the present invention are special nutrition foods (for example, milk formulas, infants and kids' foods), eatable processed foods, fish flesh goods, tobu, starch gel, noodle (for example, ramen, noodle), health supplemental foods, flavoring foods (for example, soy sauce, soy bean paste, red pepper paste, mixed paste, sauces, candy (for example, snacks), milk products (for example, fermented oil, cheese), other processed foods, kimchi, salt soaked foods (various kimchi, soy bean paste-soaked vegetable), beverage (for example, fruits, vegetable, beverage, tobu, fermented beverage, ice cream, etc.), natural flavoring sauces (for example, dried ramen soup, etc.), vitamin mixtures, alcohol beverage, drinks and other health supplemental foods. The above mentioned lists are not limited thereto. The foods, beverage or food additives might be manufactured by an ordinary method.

The term “functional foods” in the present invention represents a food group value-added to make sure that the function of a corresponding food can be more promoted from its original function with the aid of a physical, biochemical or biological engineering method or a processed food which is designed to have more of the body control function such as a biological prevention rhythm, a disease prevention and recovery that a food composition has. Preferably, it represents a food which helps prevent and improve hypertension. The functional food can include a food supplemental additive which is allowable on a food science basis and can further include a carrier, an excipient and diluent.

The term “beverage” in the present invention represents a thing which can be drinkable for killing thirst or for tasting it while including functional beverage. The beverage contains as necessary components the hydrolysate or peptides of the present invention mixed at a certain set ratio, and other components are not limited. Various flavoring agents or natural carbohydrate can be added as additive components. The natural carbohydrate is monosaccharides, disaccharide for example glucose and fructose, for example maltose, sucrose and polysaccharides, ordinary sugar such as dextrin, cyclodextrin and sugar alcohol such as xylitol, sorbitol, erythritol. In addition to them, a flavoring agent is a natural flavoring agent (thaumatin, stevia (for example, rebaudioside A, glycyrrhizin) and synthetic flavoring agent (saccharin, aspartame). The ratio of the natural carbohydrate is 1 to 20 g per 100 ml of the composition of the present invention, and preferably 5 to 12 g. The composition of the present invention might further contain a fruit flesh used to manufacture natural fruit juice, fruit juice beverage or fruit beverage.

The composition of the present invention might contain various nutritional supplements, vitamin, minerals (electrolyte), flavoring agents such as combined flavoring agents and natural flavoring agents, coloring agents and fillers (cheese, chocolate), pectic acid and salt of it, alginic acid and the salt of it, organic acid, protective collide thickener, pH adjuster, stabilizer, antiseptic, glycerin, alcohol and carbonization agent used for carbonated beverage. The above listed components can be independently used or can be used in combination.

The terms “functional beverage” in the present invention represents a beverage group processed to have a value-added function depending on the function of a corresponding beverage with the aid of a physical, biochemical and biological engineering technology or a beverage processed to have further functions with respect to the body control functions such as a biological prevention rhythm control, a disease prevention and recovery.

The present invention is directed to a hypertension therapy method comprising a step for medicating, to an object except for a human being, a hydrolysate of a gelatin extract isolated from the skins of a skate ray or a peptide formed of an amino acid sequence of MVGSAPGVL (molecular weight: 829 Da) or LGPLGHQ.

The hydrolysate of a gelatin extract isolated from the skins of a skate ray and the peptide formed of an amino acid sequence of MVGSAPGVL (molecular weight: 829 Da) or LGPLGHQ can be used in manufacturing the hypertension therapy medicine and the health functional foods as an active ingredient having the prevention, therapy and improvements effects in terms of hypertension.

The present invention will be described in more details along with the embodiments. It is obvious to an ordinary person skilled in the art that the embodiments below are provided for describing the present invention in details, not for limiting the scope of the present invention.

### Embodiment 1

### Preparation of Gelatin Extract from Skins of Skate Ray

The skins of a skate ray (Okamejei kenojei) were purchased from Youngsanhongeo Corporation and were washed with water so as to remove the impurities from the skins and were cut with scissors into pieces of 3 cm×1 cm, and the cut pieces were soaked in 1% Ca(OH)₂ for 3 days so as to remove the impurities such as proteins except for subcutaneous fat and collagen. Afterward, it is simply washed, and water mounting to 4 times of the volume of the cut skins of a skate ray was added to the water bath, and the hot water extraction was performed at pH 6.0 at a temperature of 65° C. for 6.5 hours, and the extract was obtained by way of centrifugation at a speed of 12,000×g for 10 minutes, and the gelatin extract was obtained from the skins of a skate ray following a desalting process and was dried and powdered.

### Embodiment 2

### Preparation of Hydrolysate from Gelatin Extract of Skins of Skate Ray

The hydrolysis was performed with respect to the gelatin extract of the skins of a skate ray obtained in the embodiment 1. The hydrolysis was performed by sequentially using two enzymes so as to perform a hydrolysis with respect to the gelatin. First of all, Alcalase enzyme (purchase of sigma) was used to react the extract at a temperature of 50° C. at pH 7 for 1 hour, and the first hydrolysis was conducted in such a way that the enzyme (Alcalase) vs. substrate (powdered gelatin extract) was mixed at a ratio of 1:100 (w/w).

Afterward, the second hydrolysis was conducted using another enzyme of Protease type X (purchase of sigma) with respect to the reactant, and they were reacted at a temperature of 37° C. at pH 7 for 4 hours, and at this time the enzyme (Protease type X) vs. substrate (powdered gelatin extract) was mixed at a ratio of 1:100 (w/w). When the hydrolysis reaction was completed, the enzyme was inactivated by heating in the heat tank for 10 minutes, and the hydrolysate was isolated from the enzyme after the inactivation of enzyme and was frozen and dried. The frozen and dried hydrolysate was stored at −80° C. before an actual use. The above described experimental conditions for the sake of the hydrolysis of the present invention are shown in Table 1.

TABLE 1
Process conditions for hydrolysate

| | Concentration | Enzyme:substrate | |
|---|---|---|---|
| | of substrate | mixing ratio | Reaction |
| Hydrolysis step | (%) | (w:w) | condition |
| First hydrolysis | 2 | 1:100 | 50° C., pH, |
| (Alcalase enzyme | | | 1 hour |
| processed) | | | |
| Second hydrolysis | 1.8 | 1:100 | 37° C., pH 7, |
| (Protease type X | | | 4 hours |
| enzyme processed) | | | |

In addition, during the hydrolysis, WALDI-TOF was performed with respect to the extract before the hydrolysis reaction, the first hydrolysate and the second hydrolysate, and the molecular weight distributions of each extract were measured. The gelatin extract of the skins of a skate ray having the molecular weight of >10 kDa, the gelatin extract of the skins of a skate ray having the molecular weight of <3 kDa and the gelatin extract of the skins of a skate ray were isolated using the molecular weight cut-off (MWCO) 10,000, 5,000, 3,000 and 1,000 Da.

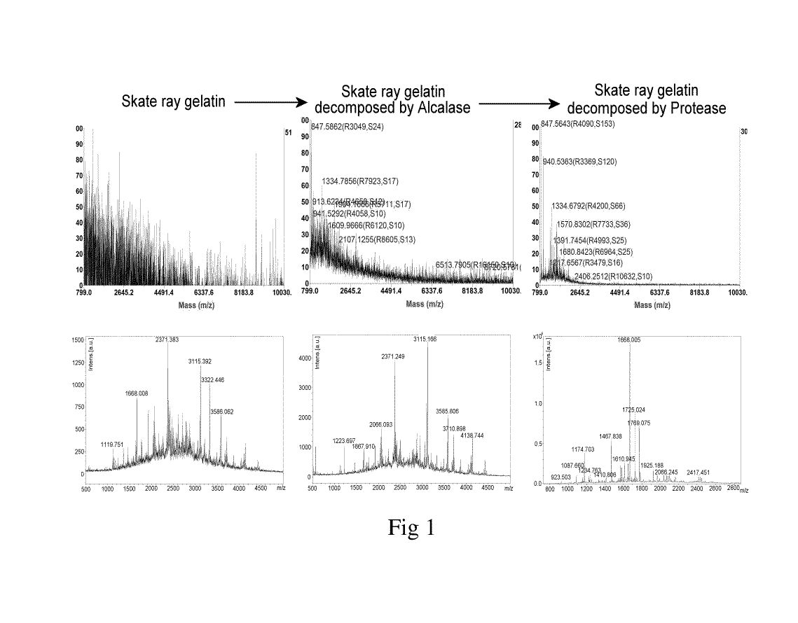

As a result of the above mentioned process and the MALDI-TOF analysis, as shown in FIG. 1, it was confirmed that the extract before the hydrolysis reaction had peaks each having a molecular weight of above >10 kDa, and the first hydrolysate after the Alcalase process had peaks each mainly having a molecular weight of <3 kDa and the second hydrolysate after the Protease type X had peaks each mainly having a molecular weight of <1 kDa.

### Embodiment 3

### ACE Inhibition Activity Measurement on Hydrolysate of Gelatin Extract Made from Skins of Skate Ray

The inventors of the present invention measured the ACE inhibition activities based on the process concentration with respect to the second hydrolysate containing a lower molecular weight among the gelatin extracts of the skins of a skate ray obtained from the above embodiments so as to develop a new hypertension therapy medicine which could be quickly absorbed by a human body and has a good effect, and the measurements were conducted in the method of measuring IC50 concentration which could inhibit 50% of the angiotensin converting enzyme in accordance with the angiotensin converting enzyme inhibition measurement method.

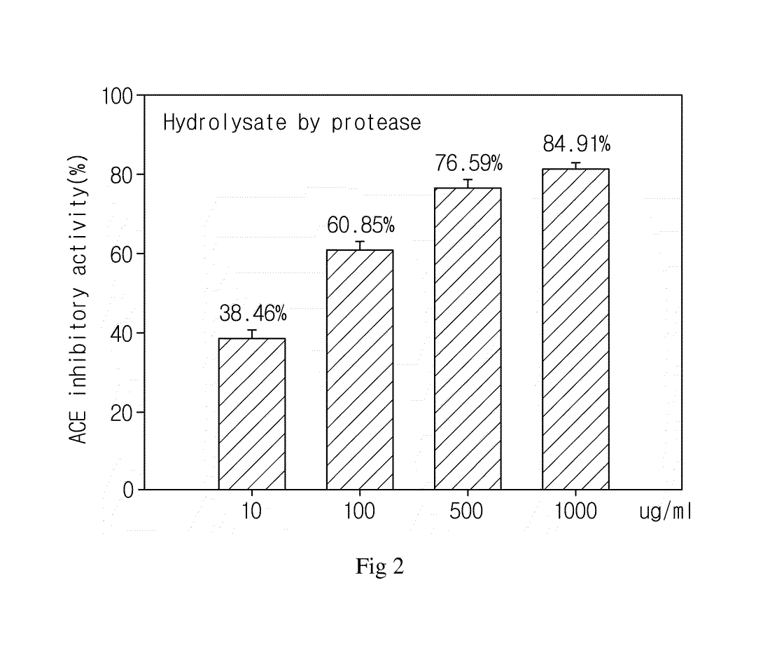

As a result, as shown in FIG. 2, it was shown that the angiotensin converting enzyme inhibition rose in proportion to the process concentration of the extract, and in particular it was 13.87 ug/ml at the concentration of IC 50. It was verified that the second hydrolysate with respect to the gelatin extract made from the skins of a skate ray had an ACE inhibition activity at a very low concentration, so the best antihypertensive activity was obtained.

### Embodiment 4

### Antihypertensive Effect Analysis on Hydrolysate of Gelatin Extract Made from the Skins of a Skate Ray with Respect to Hypertension Animal Models

The apriority hypertension rats were purchased for the sake of experiments from the animal experiment and research center of Kunkuk University. The second hydrolysate obtained from the embodiment with respect to the hypertension rats was fed to the rats with an amount of 500 mg/kg and 1000 mg/kg for 20 days, and the groups that the second hydrolysate was not fed were used as the comparison group. The positive control group was medicated with captopril known as an antihypertensive medicine (angiotensin converting enzyme inhibition agent). Afterward, the number of heart beats of each group were countered at after 2 hours, 24 hours, 72 hours, 10 days and 20 days, and the SBP (Systolic Blood Pressure) was measured. The pressure of the artery at the tail of the rat was measured by the tail cuff method. In addition, the SHR rat models were inputted into the correction frame for the purpose of measuring the blood pressures under more stable conditions, and the blood pressure was repeatedly measured at least 5 times, and the average value was recorded with the highest and lowest values being removed.

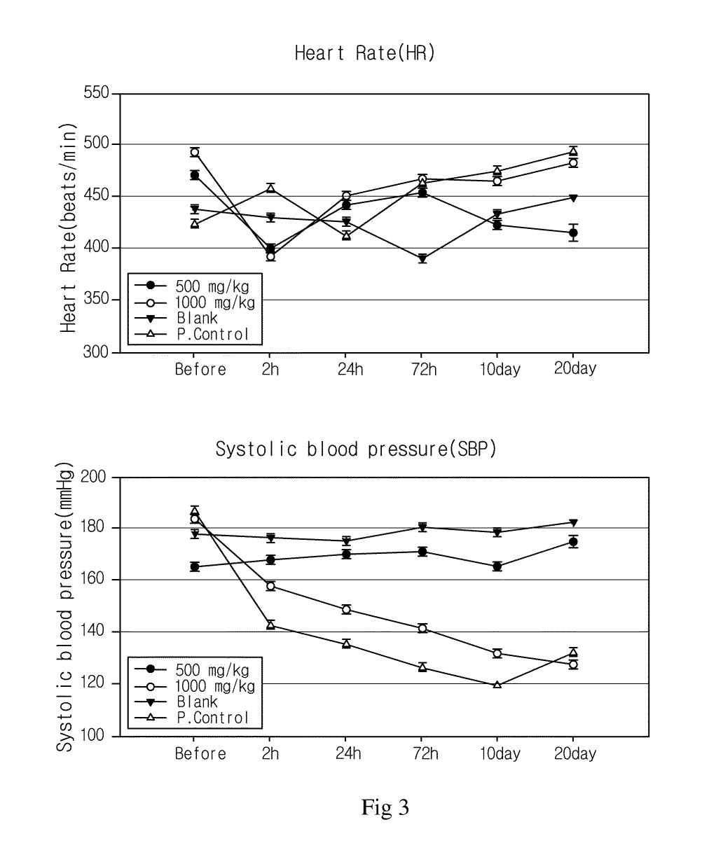

As a result, as shown in FIG. 3, there was not a big difference in the heart beat counts as compared to when the experiment was not performed; however when the second hydrolysate was taken with the amount of 1000 mg/kg, it was shown that the change of the systolic phase blood pressure was similar with the captopril-processed group which was the positive control group.

In addition, the inventors of the present invention measured the average and diastolic phase blood pressure with respect to the experimental animal groups, and the blood pressure of the rain artery was measured by the tail cuff method. The SHR rat models were inputted into the correction frame so as to measure the blood pressure under the stable conditions, and it was repeatedly performed at least 5 times, and the average value was recorded with the highest and lowest values excluded. As a result, the group fed with the second hydrolysate by 500 mg/kg showed a slight change in the average and diastolic phase blood pressure change, and the group fed with the second hydrolysate by 1000 mg/kg featured in that the average and diastolic phase blood pressure were similar with the captopril-processed group. In addition, considering that the hypertension standard level is SBP>140 mmHg/DBP>90 mmHg, it was verified that the systolic phase and diastolic phase blood pressures recovered to the normal values 3rd days after the second hydrolysate was fed according to the present invention.

So, the inventors of the present invention have confirmed that the hydrolysate (specifically speaking, the second hydrolysate) made from the gelatin extract of the skins of a skate ray shows the pharmaceutical effects similar with the captopril which is one of the antihypertensive medicines, which means that the captopril can be substituted with a natural hypertension therapy medicine, in other words, the hydrolysate of the gelatin extract of the skins of a skate ray according to the present.

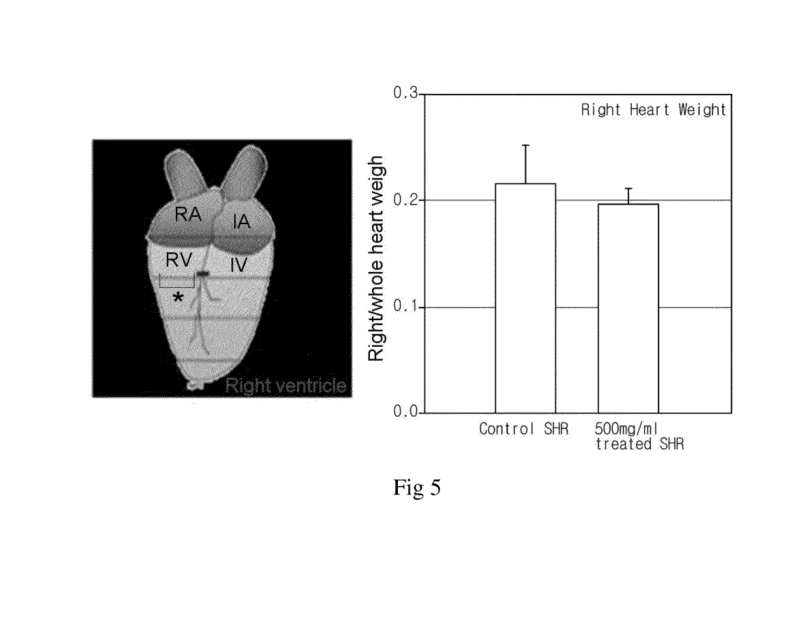

The inventors of the present invention measured the weights of the right ventricle by extracting it from the dead rats. As a result, the right ventricle of the SHR which grew fatty owing to hypertension lost the weight due to the medication of the second hydrolysate. (refer to FIG. 5).

### Embodiment 5

### Refinement of Peptide with Antihypertensive Activity

The inventors of the present invention isolated two active peptides by way of FPLC (Fast Protein Liquid Chromatography) with respect to the second hydrolysate so as to isolate the functional peptides having an antihypertensive activity contained in the second hydrolysate of the gelatin extract obtained from the skins of a skate ray. As a result of the analysis on the base sequence of the peptide, it was confirmed that the peptide had the following base sequence. In addition, the ACE inhibition (IC50) was checked in the earlier explained method so as to check the antihypertensive activity with respect to the peptides.

In addition, the determination of the sequence of the peptide was performed based on the following experiment. The objects obtained by way of the column was fully dried in the micro-centvac. The dried samples were dissolved in the distilled water containing 0.1% TFA of 20 ul, and it was activated with 50% acetonitrile so as to remove the salts from the sample and was loaded on the ZipTip C18 (Pierce #87782) balanced with the distilled water containing 0.1% TFA. It was washed with the distilled water with 0.1% TFA 2 or 3 times, and the peptide coupled to ZipTip C18 was processed to elute a 70% acetonitrile containing 0.1% TFA. The eluted peptide solution was loaded by 10 uL on the micro filter preprocessed with the biobrene AB systems Co., Ltd. USA) and was dried with argon gas.

The dried filter was mounted on the cartridge, and the amino acid sequence was determined by way of the pulsed liquid method of AB1492 automated protein sequencer, applied biosystem, Forster, C.A. USA.

In addition, the molecular weights and the decomposed ions were detected using the mass analyzer using the electro-spray ionization (ESI) method, and the refined peptide molecular weight was checked. The mass spectrum was obtained by the data dependant MS/MS method using the nano-ESI interface on the mass analyzer Q-TOF2 (Mircormass, U.K).

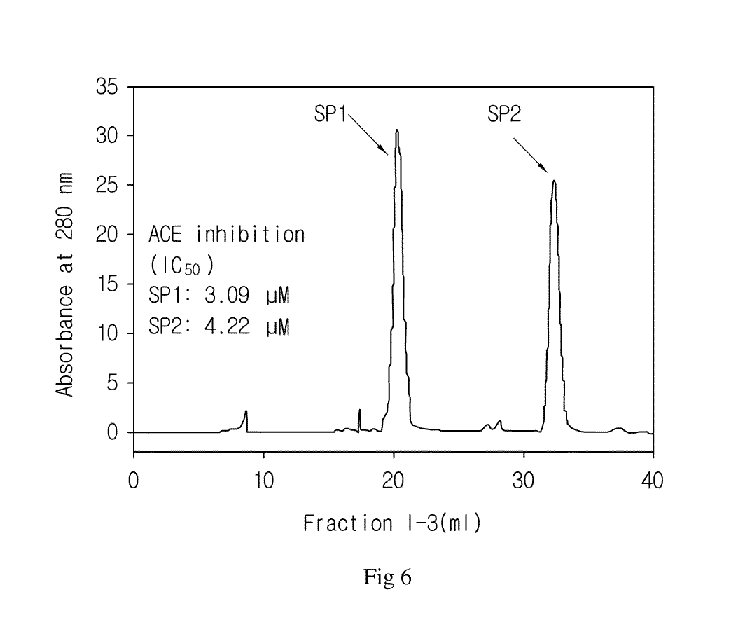

As a result, the isolated two peptides were proved to be SPI (active peptide 1)=MVGSAPGVL (829 Da), SP2 (active peptide 2)=LGPLGHQ (720 Da). As shown in FIG. 6, it was proved that the two peptides all have the ACE inhibition, and in particular the SPI (active peptide I) peptide was proved to have a higher antihypertensive activity as compared to the SP2 (active peptide 2).

So, the inventors of the present invention have confirmed that the second hydrolysate of the gelatin extract made from the skins of a skate ray and the two peptides of MVGSAPGVL and LGPLGHQ isolated from the hydrolysate and purified can be used for the sake of medicines for a hypertension therapy and as the materials of functional foods.

### Formulation Example 1

### Preparation of Pharmaceutical Formulation

### <1-1> Preparation of Powder

| | |
|---|---|
| Second hydrolysate or peptide of the present invention | 2 g |
| lactose | 1 g |

The above components are mixed and filled in a sealed pack, thus preparing the powders.

### <1-2> Preparation of Tables

| | |
|---|---|
| Second hydrolysate or peptide of the present invention | 100 mg |
| corn starch | 100 mg |
| lactose | 100 mg |
| stearic acid magnesium | 2 mg |

The above components are mixed and tabled-formed by a conventional tablet manufacture method, thus preparing the tablets.

### <1-3> Preparation of Capsules

| | |
|---|---|
| Second hydrolysate or peptide of the present invention | 100 mg |
| corn starch | 100 mg |
| lactose | 100 mg |
| stearic acid magnesium | 2 mg |

The above components are mixed and filled into the gelatin capsule by a conventional capsule manufacture method, thus preparing the capsules.

### <1-4> Preparation of Pills

| | |
|---|---|
| Second hydrolysate or peptide of the present invention | 1 g |
| lactose | 1 g |
| glycerin | 1 g |
| xylitol | 0.5 g |

The above components are mixed and formed in a pill shape with 4 g per one

### Formulation Example 2

### Preparation of Foods

The health foods were prepared as follows.

### <2-1> Preparation of Drinks

| | | |
|---|---|---|
| Honey | 522 | mg |
| Thioctic Acid | 5 | mg |
| nicotinic acid amide | 10 | mg |
| hydrochloric acid riboflavin potassium | 3 | mg |
| pyridoxine hydrochloride | 2 | mg |
| inositol | 30 | mg |
| orotic acid | 50 | mg |
| Second hydrolysate or peptide of the present invention | 1.28 | mg |
| water | 200 | ml |

### <2-2> Preparation of Candy

| | | |
|---|---|---|
| sugar | 50~60 | weight % |
| starch syrup | 39.26~49.66 | weight % |
| Second hydrolysate or peptide of the present | 0.24~0.64 | weight % |
| invention | | |
| orange flavor | 0.1 | weight % |

The candies were prepared with the above composition and contents in a conventional method.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

1. A method of preventing and treating hypertension comprising administering to a subject in need thereof a composition comprising a hydrolysate of a gelatin extract isolated from the skins of a skate ray, the hydrolysate being contained as an active ingredient.

2. The method of claim 1, wherein the hydrolysate is a second hydrolysate which is obtained by way of a first hydrolysis with Alcalase with respect to the gelatin extract and then by way of a hydrolysis with Protease type X.

3. The method of claim 2, wherein the gelatin extract is obtained in such a way that the skins of a skate ray are soaked in 1% of Ca(OH)₂ and is hot water extracted at pH 6.0 and a temperature of 60˜70° C. for 6˜7 hours.

4. The method of claim 2, wherein the second hydrolysate includes a peptide formed of an amino acid sequence of MVGSAPGVL (molecular weight: 829 Da) or LGPLGHQ (molecular weight: 720 Da).

5. The method of claim 1, wherein the hydrolysate is contained with a concentration of 10 ug/ml˜1500 ug/ml in the composition.

6. A method of preventing and treating hypertension comprising administering to a subject in need thereof a peptide formed of an amino acid sequence of MVGSAPGVL (molecular weight: 829 Da) or LGPLGHQ (molecular weight: 720 Da), the peptide being contained as an active ingredient.

7. The method of claim 6, wherein the peptide is isolated from a hydrolysate of a gelatin extract isolated from the skins of a skate ray.

8. A method for preparing a hydrolysate of a gelatin extract made from the skins of a skate ray having an antihypertensive activity, comprising:
a step for obtaining a gelatin extract from the skin of a skate ray;
a step for performing a first hydrolysis by adding an Alcalase enzyme to the gelatin extract; and
a step for performing a second hydrolysis by adding a Protease type X enzyme to the first hydrolysate.

9. The method of claim 8, wherein the gelatin extract is obtained in such a way that the skins of a skate ray are soaked in 1% of Ca(OH)₂ and is hot water extracted at pH 6.0 and a temperature of 60˜70° C. for 6˜7 hours.

10. The method of claim 8, wherein the first hydrolysis is performed in such a way that Alcalase vs. substrate (gelatin extract) is mixed at a ratio of 1:100 (w:w) and is reacted at pH 7.0 at a temperature of 45˜55° C. for 1 to 2 hours.

11. The method of claim 8, wherein the second hydrolysis is performed in such a way that Protease type X vs. substrate (gelatin extract) is mixed at a ratio of 1:100 (w:w) and is reacted at pH 7.0 at a temperature of 35˜40° C. for 3 to 6 hours.

12. The method of claim 8, wherein the second hydrolysate obtained by way of the second hydrolysis contains peptide the molecular weight is less than 1 kDa.

13. The method of claim 12, wherein the peptide has an amino acid sequence of MVGSAPGVL (molecular weight: 829 Da) or LGPLGHQ (molecular weight: 720 Da).

14. A health function food for a hypertension prevention and improvement, comprising:
a hydrolysate of a gelatin extract made from the skins of a skate ray; and
a peptide formed of an amino acid sequence of MVGSAPGVL (molecular weight: 829 Da) or LGPLGHQ.

15. The food of claim 14, wherein the hydrolysate is contained with a concentration of 100 ug/ml˜1500 ug/ml.
